(19) United States
(12) Patent Application Publication
(10) Pub. No.: US 2014/0316411 A1
(43) Pub. Date: Oct. 23, 2014

(54) SYSTEMS AND METHODS FOR MINIMALLY INVASIVE FRACTURE REDUCTION AND FIXATION

(71) Applicant: Beth Israel Deaconess Medical Center, Boston, MA (US)

(72) Inventor: Charles S. Day, Chestnut Hill, MA (US)

(73) Assignee: BETH ISRAEL DEACONESS MEDICAL CENTER, Boston, MA (US)

(21) Appl. No.: 14/358,955

(22) PCT Filed: Nov. 16, 2012
(86) PCT No.: PCT/US2012/065529

§ 371 Date: May 16, 2014

Related U.S. Application Data
(60) Provisional application No. 61/561,099, filed on Nov. 17, 2011.

(51) Int. Cl. A61B 17/72

(52) U.S. Cl. CPC A61B 17/7225
USPC 606/63

(57) ABSTRACT

Systems and methods of using expandable elements inserted into a bone, such as the distal radius, to provide for minimally invasive reduction and fixation of fractures. An introducer is used to insert the expandable elements to create a cavity within the bone, to precisely reposition displaced bone fragments and to form a cavity for introduction of an implant material for bone fixation.

### CROSS REFERENCE TO RELATED APPLICATIONS

This application claims the benefit of U.S. provisional patent application No. 61/561,099 filed on Nov. 17, 2011, which is incorporated herein in its entirety by reference.

### BACKGROUND OF THE INVENTION

Distal radius fractures are a common fracture of the upper extremity. Reduction is a medical procedure to restore the correct alignment of displaced bone fragments, either with or without surgery. Secondary displacement of fracture fragments can occur over time during treatment, while the fracture is healing. Existing methods for the reduction of complex bone fractures of the radius typically require the use of wires, plates and screws to stabilize the bone fragments so that healing can occur. However, the small size of the fragments and the occurrence of tendon irritation associated with the use of these techniques can cause discomfort and impair healing. Consequently, further improvements in devices and methods for treating more complex fractures are needed.

### SUMMARY OF THE INVENTION

The present invention relates to systems and methods of using expandable elements inserted into bones such as the distal radius to provide for the reduction and stabilization of fractures. A substantial proportion of the fracture of the distal radius involves intra-articular bone factures that result in greater difficulties in achieving reduction. Preferred embodiments of the present invention relate to systems and methods for obtaining reduction of intra-articular bone fragments using expandable elements inserted into bones such as the distal radius.

Preferred embodiments of the invention utilize a first expandable member such as a balloon inserted along the fracture of the distal radius using a delivery system with an introducer device such as a cannula. The delivery system can be inserted percutaneously or the surgeon can expose the fracture site by mini-incision (about 1 cm). The expandable element is expanded to define a cavity in which material can subsequently be inserted to form a rigid implant. As only a single small hole is formed in the cortical bone, there is minimal trauma involved in the insertion of the expandable components.

A preferred embodiment of the invention can utilize a second expandable member such as a balloon that is inserted into the first expandable member to maintain the reduction during insertion of the implant material. Thus, an inner element is used to stabilize one or more fragments during implant formation. Preferred embodiments of the invention further include removal of the first and/or second expandable members during or after implant formation.

Systems in accordance with the invention include a minimally invasive delivery system for introduction of the first and second expandable members, such as balloons, into the fractured bone, a stabilizing device to stabilize the delivery system relative to the fracture, a fluid delivery system for balloon expansion and removal, and an implant delivery system to deliver an implant material into the fracture using the delivery system. The delivery system can be anchored into position relative to the facture location thereby enabling the user to insert tools through the delivery system into a region at or adjacent to a fracture to move bone fragments into proper position. The delivery system includes an introducer that enables fluid delivery into the inflatable or expandable components positioned within the bone. The components of the surgical system can be packaged as a kit. The system can also be used for other joint bones and structures within the human body.

### DESCRIPTION OF THE DRAWINGS

The invention will be more fully understood from the following detailed description taken in conjunction with the accompanying drawings in which:

FIG. 1A is a schematic illustration of an embodiment of an outer balloon apparatus of a device for assisting in the reduction of a distal radius fracture;

FIG. 1B is a schematic illustration of an inner balloon apparatus of the device in use with the outer balloon apparatus of FIG. 1A;

FIG. 2A is a schematic illustration of the inner balloon apparatus with one embodiment of an inner balloon;

FIG. 2B is a schematic illustration of the inner balloon apparatus with a further embodiment of an inner balloon;

FIG. 2C is a schematic illustration of the inner balloon apparatus with a still further embodiment of an inner balloon;

FIG. 3A is a schematic cross sectional view of a distal radius fracture;

FIG. 3B is a schematic cross sectional side view of the fracture of FIG. 3A;

FIG. 4A is a schematic illustration of the outer balloon apparatus and an inflated outer balloon to reduce the fracture;

FIG. 4B is a side view of FIG. 4A;

FIG. 5A is a schematic illustration of an inflated inner balloon to further reduce the fracture;

FIG. 5B is a side view of FIG. 5A;

FIG. 6A is a schematic illustration of a bone-filling material introduced into the void left by the ruptured outer balloon, while the inner balloon remains in place;

FIG. 6B is a side view of FIG. 6A;

FIG. 7A is a schematic illustration of the deflation and withdrawal of the inner balloon from the cavity;

FIG. 7B is a schematic illustration of a bone-filling material introduced into the void left by the inner balloon;

FIG. 8 is a schematic illustration of an outer balloon apparatus stabilized with external pins;

FIG. 9 is a schematic illustration of a further embodiment including a slidable sleeve for withdrawal of an outer balloon;

FIG. 10 is a schematic illustration of a further embodiment with multiple cannulas;

FIGS. 11A-11B are schematic illustrations of an intra-articular comminuted A-O Type C distal radius fracture;

FIGS. 12A-12B are schematic illustrations of an inflated inner balloon shaped to further reduce the fracture of FIG. 11;

FIG. 13 is a schematic illustration of a further embodiment employing multiple inner balloons in the reduction of a tibial plateau fracture;

FIG. 14A is a schematic illustration of an embodiment of a probe apparatus in a collapsed state for use with an outer balloon apparatus;

FIG. 14B is a schematic illustration of the probe apparatus of FIG. 14A in an expanded state; and

FIG. 15 is a schematic illustration of a kit with a device for the reduction of a fracture.

### DETAILED DESCRIPTION OF THE INVENTION

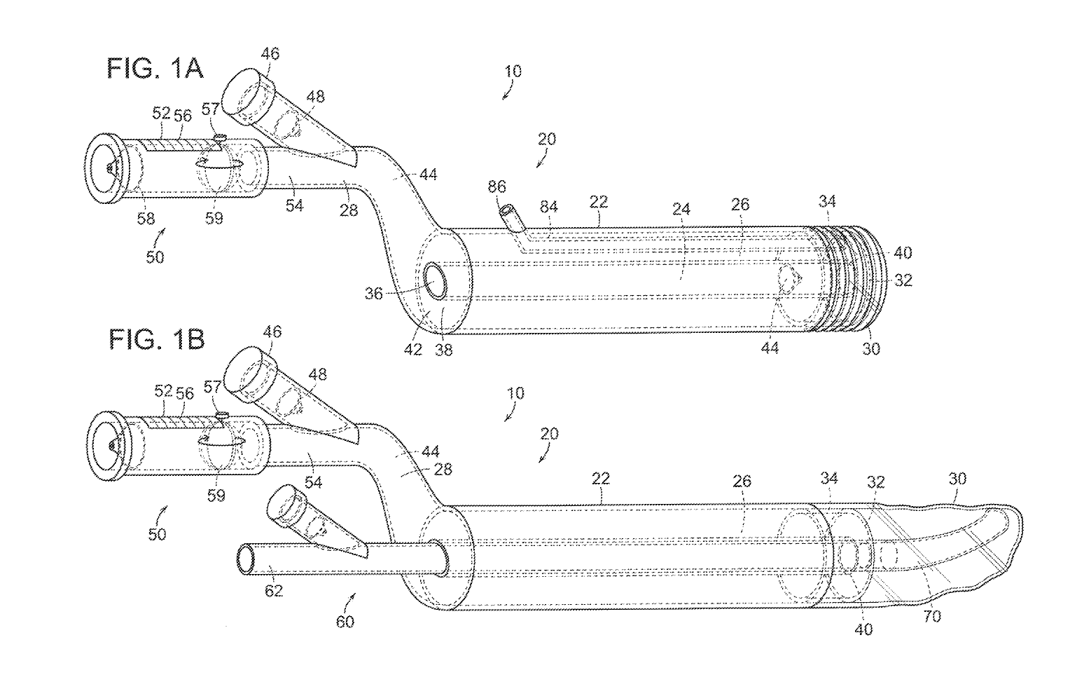

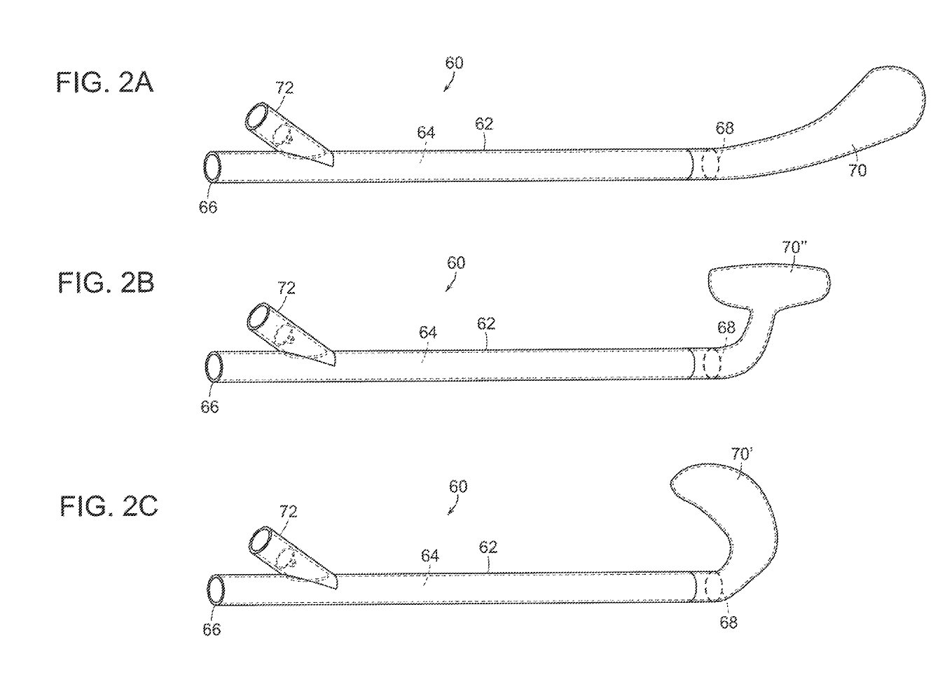

An embodiment of a delivery system 10 for assisting in the reduction of a fracture, such as a distal radius fracture, is illustrated in FIGS. 1A-1B and 2A-2C. The delivery system 10 includes an introducer device including an outer introducer element, such as an outer balloon apparatus 20, and an inner introducer element, such as an inner balloon apparatus 60.

The outer balloon apparatus includes a support element for mounting an outer expandable member, such as an outer balloon 30, and provides for the insertion and inflation of the outer balloon within the distal radius to provide compaction of bone fragments and an initial reduction of the bone. The inner balloon apparatus 60 includes a support element for mounting an inner expandable member, such as an inner balloon, and fits within at least a portion of the outer balloon apparatus and provides for the insertion and inflation of a smaller, inner balloon 70, within the inflated outer balloon, to allow more precise control of the reduction. While the inflated inner balloon maintains the reduction, the outer balloon is ruptured at a preset weakened region by overinflation, for example. A flowable, bone-filling material is introduced into the void left by the outer balloon, exterior to the inner balloon. When the bone-filling material has at least partially cured and solidified, the inner balloon can be deflated and either withdrawn or detached from the inner balloon apparatus, and the smaller void left by the inner balloon can also be filled with a bone-filling material.

More particularly, in the embodiment illustrated in the figures, the outer balloon apparatus 20 includes a longitudinally-extending tubular portion 22 having an inner cannula 24 and an annular outer cannula 26 coaxially arranged around the inner cannula. The outer balloon apparatus also includes a junction portion 28 connected to the tubular portion 22, to provide fluid communication with the outer cannula 26. An outer balloon 30 mounts to a mounting element at the distal end 32 of the tubular portion 22.

The outer balloon 30 is a bladder-like receptacle formed from a flexible, membranous elastomeric material having an opening through which a fluid can be introduced into the interior of the balloon. The outer balloon is attached to the distal end of the tubular portion in any suitable manner that allows insertion of the balloon into the distal radius through an opening in the bone. In one embodiment, a lip around the opening of the balloon can be stretched slightly to fit around the exterior of the distal end of the tubular portion. The tubular portion at the distal end may include a recessed section 34 of lesser diameter so that the balloon does not extend beyond the circumferential extent of the tubular portion. The outer balloon can be folded or rolled or otherwise collapsed on the distal end (indicated schematically in FIG. 1A), so that the balloon may be more readily inserted into a bone, as discussed further below. In another embodiment, the lip of the balloon is attached within an annular depression in an end face of the distal end of the tubular portion. In another embodiment the lip can be clamped around the distal end of the tubular portion with a clamping device.

The inner cannula 24 extends from an opening 36 at a proximal end 38 to an opening 40 at the distal end 32. The inner cannula can be supported along the longitudinal axis of the tubular portion by a support structure 42 or fitting at the proximal end. A one-way or check valve 44 is disposed within the inner cannula, preferably at a location near the distal opening, to prevent flow of fluid through the inner cannula in a direction from the distal end toward the proximal end. The one-way valve is also configured to allow the inner balloon apparatus to pass through. A steering element can be used to aid in positioning of the inner balloon.

The junction portion 28 of the outer balloon apparatus 20 includes a passage 44 therethrough that fluidly communicates with the coaxial outer cannula 26. The junction portion includes an inlet port 46 through which a fluid or a flowable material can be introduced into the outer cannula. A one-way or check valve 48 is preferably disposed below the inlet port to prevent back flow out of the port. Any suitable fitting, as known in the field, can be used as the inlet port.

The junction portion 28 also includes a pressure monitoring mechanism 50 including a pressure gauge 52 to monitor pressure within an outer balloon when connected to the outer cannula, described further below. In the embodiment shown, the pressure monitoring mechanism includes a spur channel 54 fluidly connected to the passage in the junction portion. The pressure gauge 52 is in fluid communication with the interior of the spur channel for monitoring the pressure in the channel and includes a visual indicator 56 on the exterior of the spur channel, by which the pressure value can also be read and monitored by the surgeon. The spur channel includes a proximal open end and can include a fitting for coupling. The pressure monitoring mechanism also includes a pressure control valve 58 in the spur channel to prevent flow out of outer cannula as long as the pressure remains below a determined value. A stop valve or tube-blocker 59 is also provided within the spur channel. The stop valve is preferably operable with a switch 57 or similar element from the exterior of the spur channel. The surgeon can actuate the stop valve to fully close off fluid passage through the spur channel, as described further below.

The junction portion may also serve as a handle for the outer balloon device and can be suitably shaped to allow gripping by a hand. Alternatively, a handle can be attached to or integrally formed with the junction portion or in another manner with the tubular portion.

The outer balloon apparatus can also include a vent element 82, to vent fluid from the outer balloon, described further below. In one embodiment, the vent element 82 may include a cannula 84 extending within the tubular portion 22 from an opening at the distal end 32 to a proximal opening in the tubular element. A fitting 86 may be provided to allow a suction or vacuum source to be connected to the vent element to assist in venting fluid within the outer balloon through the vent element.

The inner balloon apparatus 60 includes a tubular element 62 that fits through the inner cannula 24 of the outer balloon apparatus 20. The tubular element includes a cannula 64 that extends from a proximal end 66 to an open distal end 68. An inlet port 72 is provided on the tubular element through which a fluid can be introduced into the cannula. The length of the tubular element 62 between the inlet port 72 and the distal end 68 is sufficient to fit within the inner cannula 24 of the outer balloon apparatus 20 with the open distal ends 32, 68 of the inner and outer apparatuses disposed at generally the same location. The inner balloon apparatus is able to rotate within the inner cannula of the outer balloon apparatus, so that the inner balloon 70 can be adjusted to a desired location. A seal such as an O-ring or a gasket can be used between the exterior of the cannula of the inner balloon apparatus and the interior of the inner cannula.

The inner balloon 70 is also a bladder-like receptacle formed from a flexible, membranous elastomeric material. The inner balloon includes an opening through which a fluid can be introduced into the interior of the balloon. The inner balloon is attached to the distal end 68 of the tubular element 62 in any suitable manner that allows insertion of the balloon through the inner cannula of the outer balloon apparatus and into the cavity in the bone that has been formed by inflation of the outer balloon. In one embodiment, a lip around the opening of the balloon can be stretched slightly to fit around the exterior of the distal end of the cannula.

Both the inner and the outer balloons can be made from any suitable medical grade, elastomeric material, such as, without limitation, a polyurethane, silicone, or nylon. The material of the balloons can expand and distend without tearing upon contact with fragments of bone. Any suitable balloon manufacturing process can be used, such as, without limitation, dip forming, blow molding, injection molding, or thermoforming.

The balloons can be preformed with a shape that generally matches the region within the bone which is to be filled. Both the inner and outer balloons can be provided in a variety of preformed shapes. The surgeon can select the particular shape to be used based on the size and shape of the fracture and surrounding bone.

The inner balloon is preferably provided in at least three distinct shapes. In a first shape, the balloon 70 includes a widened portion extending in a distal direction (as shown in FIG. 2A). In a second shape, the balloon 70′ includes a widened portion extending in a proximal direction (as shown in FIG. 2C). In a third shape, the balloon 70″ includes a widened portion extending both distally and proximally (as shown in FIG. 2B). This variety of shapes gives the surgeon wide flexibility in directing the inner balloon toward the region or regions where the pressure from the inner balloon is most needed.

The preset weakness can be formed in the outer balloon, for example, where a wall section can be formed from a thinner material or a different type of material. In another example, the balloon can be formed with a rupturable seam that is held closed by, for example, an adhesive. In a further example, the material of the wall section can be pretreated, such as with a heat treatment or a chemical treatment. In yet a further example, the wall can be formed with a reinforced material in which the weakened region has no or less reinforcing. One or more weakened regions can be provided if desired. The weakened region can be in the form of a line or lines, or in the form of an area. The burst or rupture pressure can be set during manufacture.

The inner balloon can also be formed with a preset weakness in a ring-shaped region adjacent the opening. This weakness provides a rupture line for detaching the inner balloon from the inner balloon apparatus, for example, by twisting the inner balloon apparatus. This feature is useful if the inner balloon, after deflation, adheres to the cured and solidified bone-filling material.

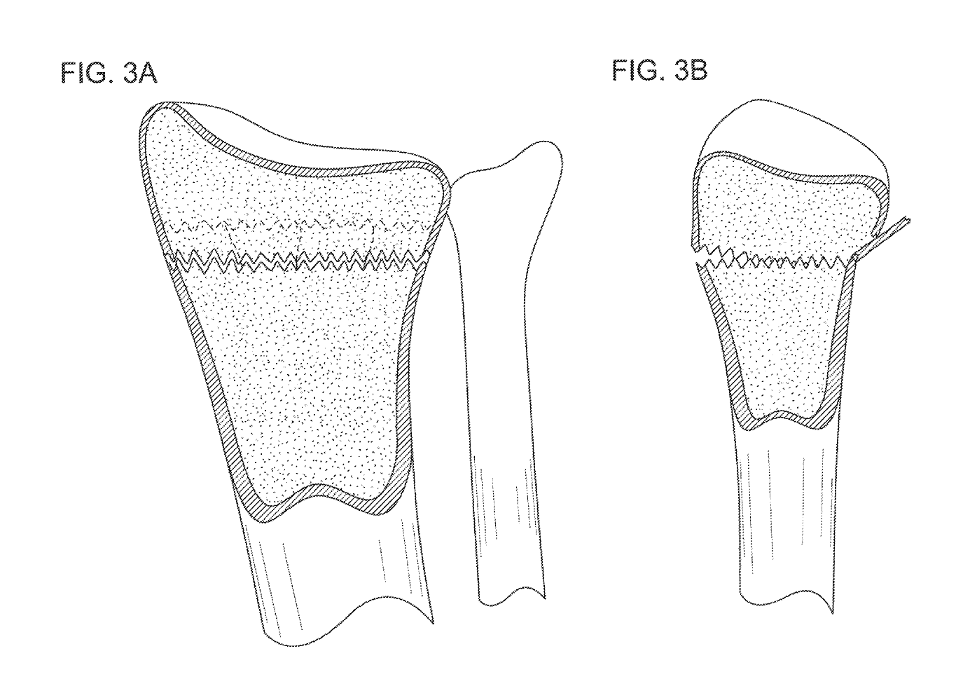

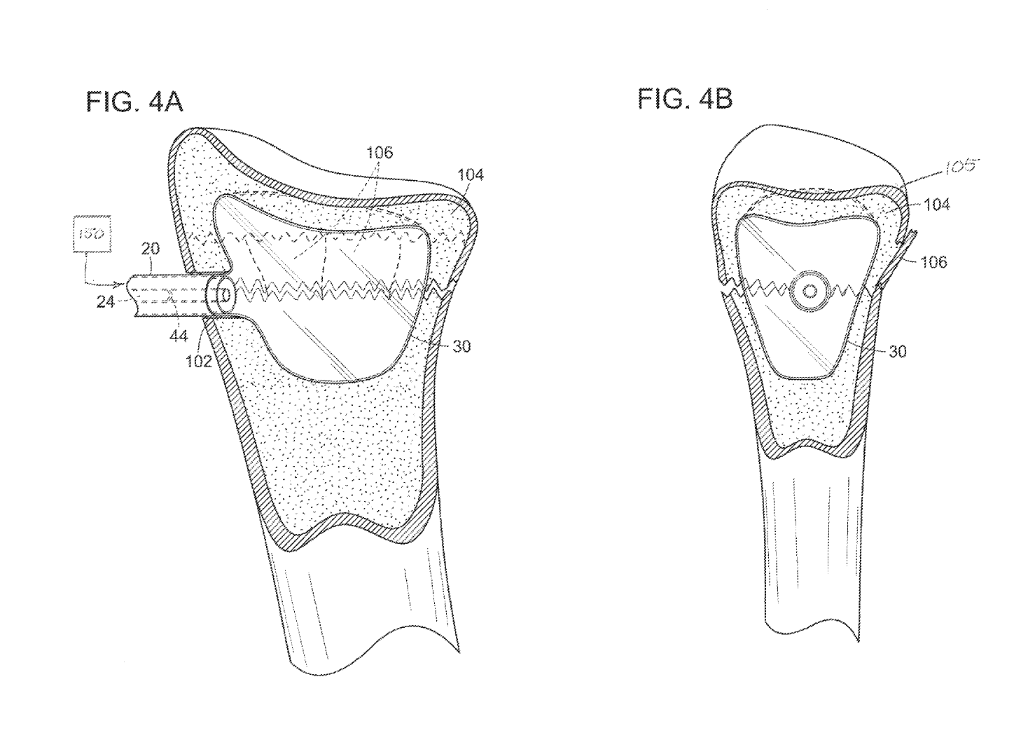

Referring to FIGS. 3A-8, a method of using the device for the reduction of a distal radial fracture is illustrated. FIGS. 3A and 3B schematically illustrate a Type A (A-O Classification) extra-articular dorsally angulated distal radial fracture. A surgeon drills a hole 102 through a single bone wall of the metaphyseal cortical bone, which is typically 1 mm in thickness, into the metaphyseal fracture line (FIGS. 4A, 4B). The drill is typically inserted about 2 mm, and the drill bit is generally at least 3.5 mm in diameter. The wrist is maintained in traction during the procedure. The outer balloon apparatus with an outer balloon attached is inserted through the drilled hole a distance sufficient to allow the outer balloon to enter the fracture. It is generally not necessary to anchor the outer introducer device, such as the outer balloon apparatus, to a distal wall portion of metaphyseal cortical bone. Thus, additional drilling with attendant further damage to the bone can be avoided.

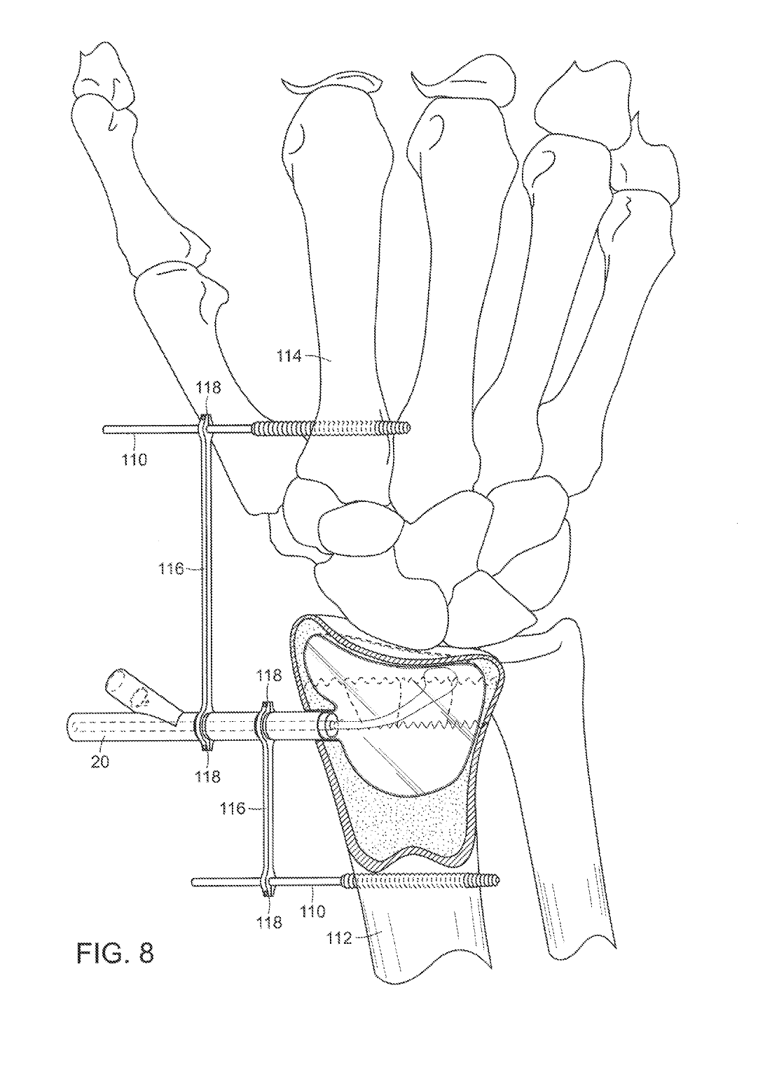

If desired, the outer balloon apparatus 20 can be anchored for stability. In one embodiment the outer balloon apparatus is fixed to one or more external screw pins 110. For example, referring to FIG. 8, a screw pin 110 is attached to the proximal cortical bone 112 of the radius, and another screw pin 110 is attached to a metacarpal bone 114. Stabilizer bars 116 extend between each pin 110 and outer balloon apparatus 20. The stabilizer bars can attach to the outer balloon apparatus and the pin in any suitable manner, such as with a pipe- or tube-type clamp fitting 118. The outer balloon apparatus can include an annular groove around the exterior surface to form a seat for the clamp fitting to prevent shifting. Alternatively, the patient's radial bone can be fixed with a rigid restraint. An armature fixed to the rigid restraint can support the delivery system.

The outer balloon 30 is inflated, for example, with fluid from a fluid source 150. See FIGS. 4A and 4B. The check valve 44 in the inner cannula prevents the inflation fluid from flowing back out of the device. The stop valve 59 in the pressure monitoring mechanism 50 is held in the open position. The pressure control valve 58 is able to release excess pressure if the outer balloon pressure nears the rupture pressure. As the balloon inflates, the cancellous bone 104 is pressed outwardly and compacted, leaving a void or cavity within the bone. The balloon can typically form a cavity with a volume of 3 to 10 cc, and more preferably 4 to 7 cc. Portion 105 distal to fragment 106 is elevated, and bone fragments such as the angulated bone fragment 106 are manipulated back into place. The surgeon views the inflation of the balloon and the manipulation of the bone fragments in real time using a suitable imaging system, such as a fluoroscopy system.

The balloon 30 can be inflated with any suitable fluid, such as air or another medical-grade gas. The surface of the balloon can be marked with radiopaque markings that can be observed on the monitor as the balloon is inflated, thereby indicating where the surface of the balloon lies. The fluid can comprise a radiopaque fluid to enable the surgeon to visually observe the inflation on the monitor of the imaging system.

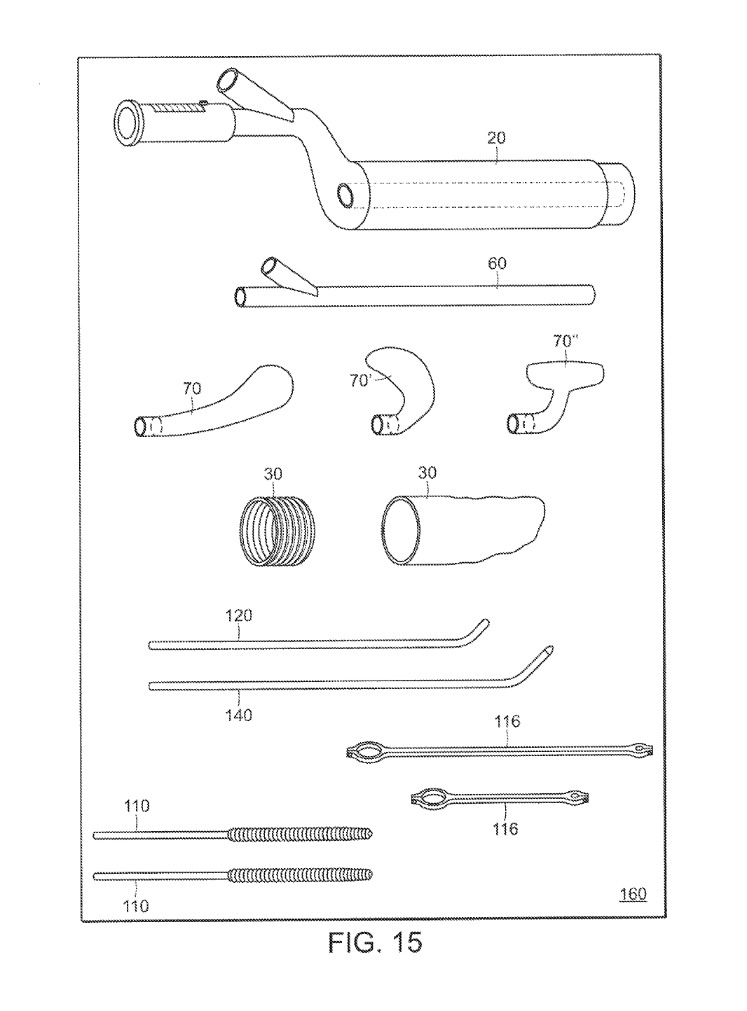

A probe or stylet 120 (see FIG. 15) can be inserted through the inner cannula 24 into the inner balloon to assist in compressing the cancellous bone and in forcing the outer balloon into the desired position within the radius. The probe can have a blunt or rounded distal tip to prevent damage to the outer balloon. The probe can be formed with a curve near the distal end, for example, using a shape memory alloy, so that the probe can be directed by the user in the desired direction.

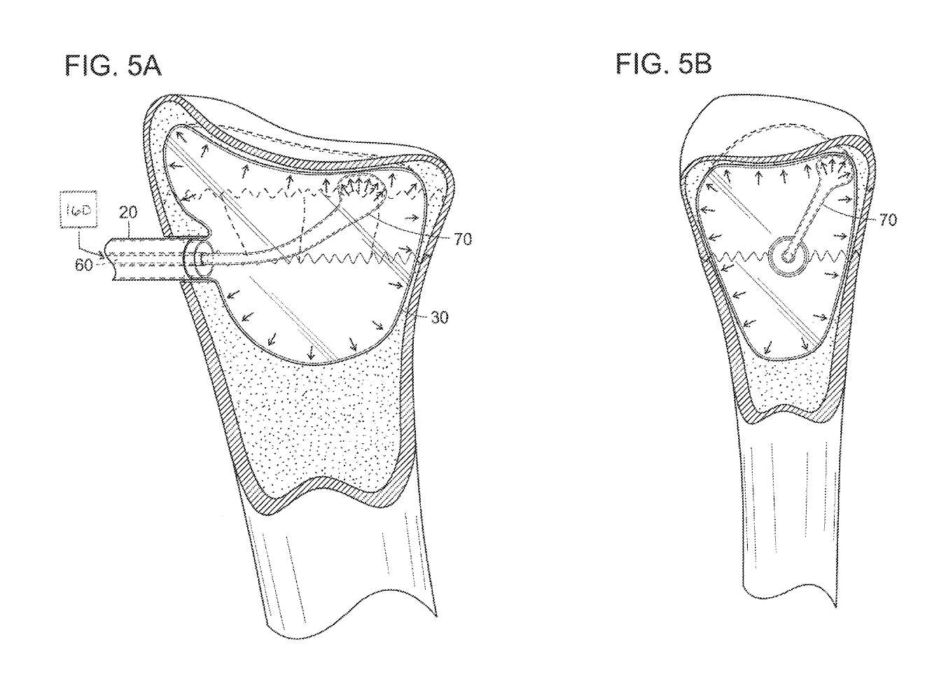

Referring to FIGS. 5A and 5B, the surgeon selects an appropriately shaped inner balloon, for example, balloon 70, and attaches it to the inner balloon apparatus 60. Alternatively, an apparatus 140 (such as shown in FIGS. 14A and 14B) can be inserted to provide additional pressure. The inner balloon apparatus 60 is then inserted through the inner cannula 24 of the outer balloon apparatus 20, past the check valve, and rotated until the balloon is placed where the extra pressure is needed to maintain the reduction. As an example, the inflation of the outer balloon has enabled a distal radial fracture (FIGS. 3A, 3B) to become better reduced, but some mild dorsal angulation is still present (FIGS. 4A, 4B). The inner balloon 70 is then inflated with fluid, for example, from a fluid source 165, which can be the same as fluid source 150. As the inner balloon 70 is inflated, the pressure within the outer balloon 30 is monitored, so that the rupture pressure of the outer balloon is not exceeded. If the pressure nears the rupture pressure, the pressure control valve actuates to release the excess pressure. Additionally, the surgeon can manually manipulate the bone fragments or fragments into their proper position through the skin.

Once reduction of the fracture is maintained by the inflated inner balloon 70 (FIGS. 5A, 5B), the stop valve 59 is held closed and the outer balloon 30 is ruptured at the pre-weakened region by over inflation of the outer balloon. Upon rupturing of the balloon, the inflation air within the outer balloon can dissipate into the bone without harm to the patient and/or vented through vent element 82. In one embodiment, the outer balloon fragment or fragments can be left in the distal radius, as the balloon is formed from medical grade, biocompatible material.

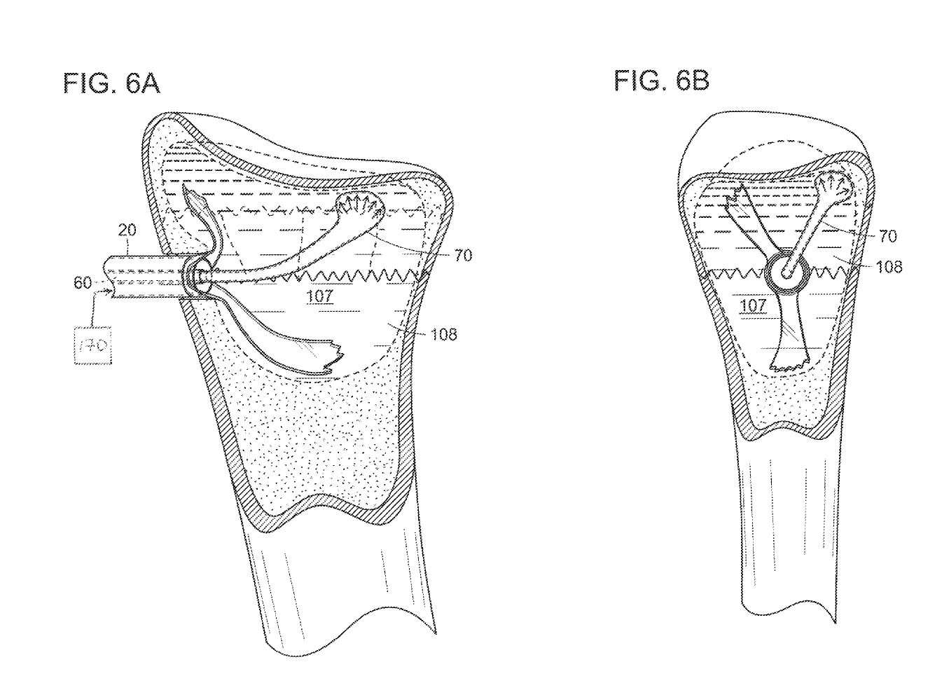

A suitable material 107, for example, from a source 170, is injected through the inlet port 46 and the outer cannula 26 into the newly-created cavity 108 left by the outer balloon (FIGS. 6A, 6B). Air or other fluid in the void can be vented through the vent element 82. A vacuum or suction source 85 can be attached to the vent element to assist in venting, if desired. The inner balloon 70 remains inflated to maintain the reduction while the bone-filling material 107 is introduced. Any suitable bone-filling material can be used. Examples include, without limitation, liquid CaSO₄, bone cement, allograft tissue, autograft tissue, or hydroxyapatite. Medications can be included in the filling material.

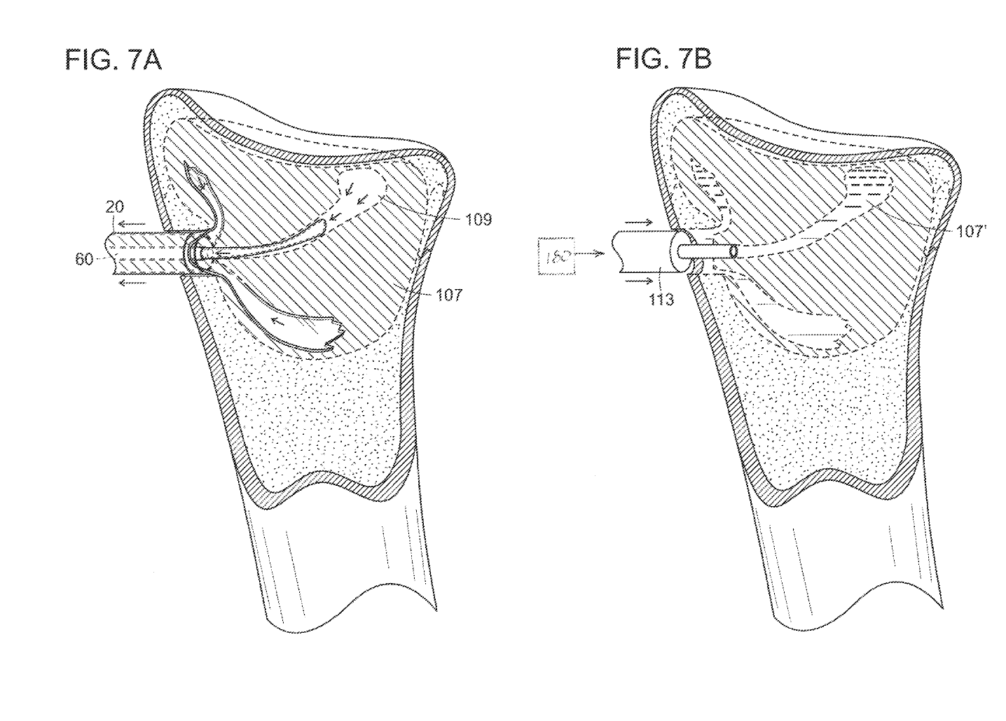

Once the bone-filling material 107 in the void 108 has cured and hardened, the inner balloon 70 can be deflated without loss of reduction. The inner and outer balloon apparatuses are removed (FIG. 7A), and the small void 109 left by the inner balloon is filled with more bone-filling material 107′, for example, by injection with a suitable injection device 113, for example, from a source 180, which can be the same as source 170 (FIG. 7B).

If the inner balloon adheres to the hardened bone-filling material, the inner balloon can be twisted off at a location close to the distal end of the cannula. The residual balloon, which is formed from a medical grade, bio-compatible material, can be left in the distal radius.

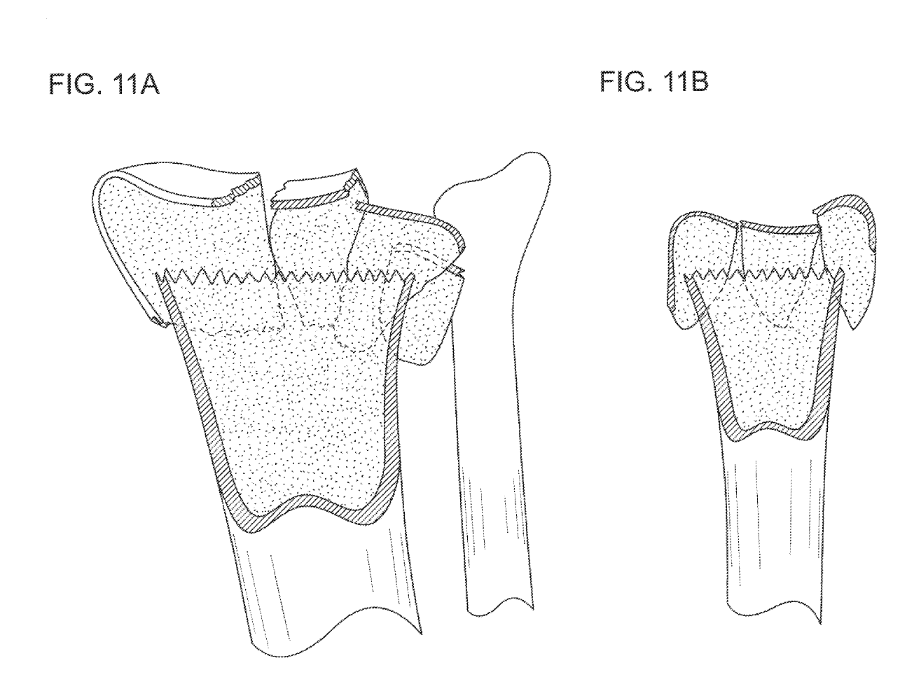

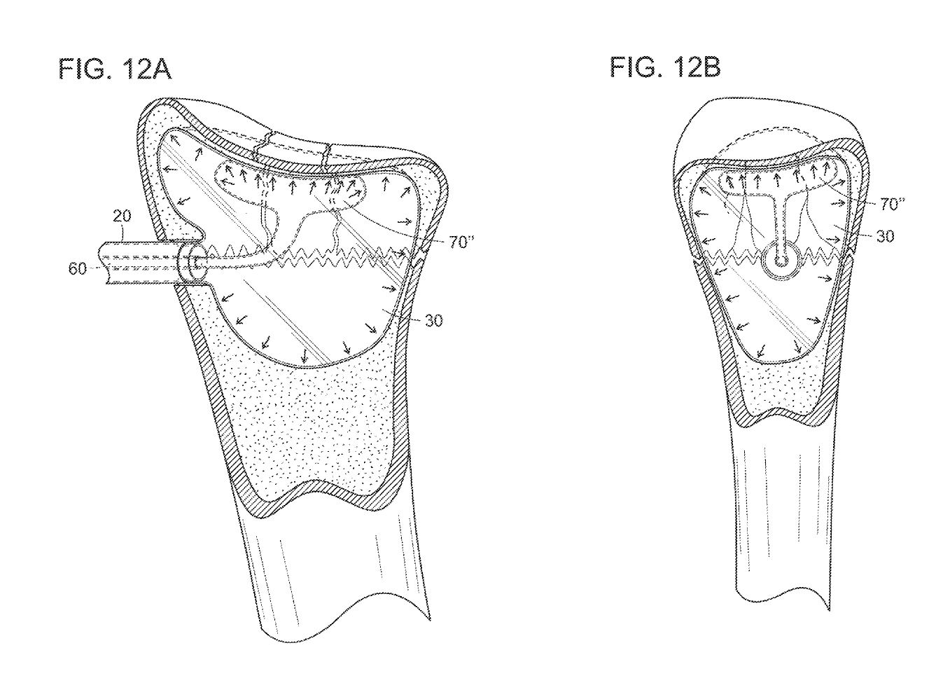

In a further example, FIGS. 11A-11B schematically illustrate orthogonal views of an intra-articular comminuted A-O Type C distal radius fracture. Referring to FIGS. 12A-12B, an inner balloon 70″, shaped to distribute pressure over a greater area (for example, the balloon of FIG. 2B), has been inserted inside an outer balloon 30 to maintain reduction of the fracture illustrated in FIGS. 11A-11B. The outer balloon can then be ruptured and a suitable bone-filling material injected into the void left by the outer balloon, as described above.

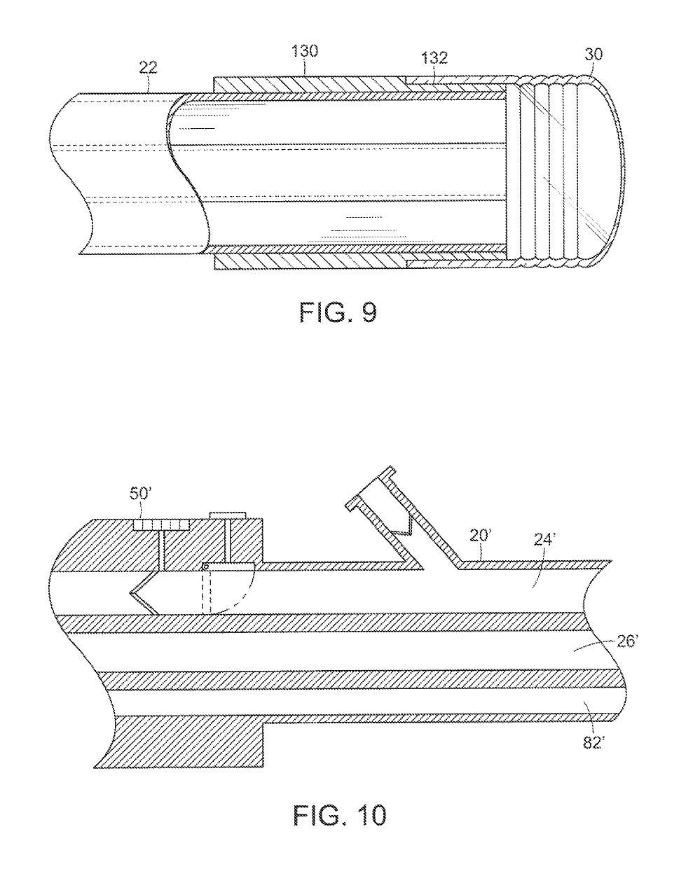

In a further embodiment the outer balloon 30 can be attached to a slideable sleeve 130 for subsequent removal of the ruptured balloon fragments as shown in FIG. 9. The sleeve is disposed circumferentially around the tubular portion 22 and can reciprocate along the tubular portion. The sleeve includes a reduced section 132 of lesser diameter to which the lip of the balloon is attached, as described above. When the balloon is ruptured, the fragments can be withdrawn by sliding the sleeve, to which the lip of the balloon remains attached, in the proximal direction.

In a still further embodiment, in an outer balloon apparatus 20′, a cannula 24′ for the inner balloon apparatus is adjacent to a cannula 26′ for introducing fluid into the outer balloon. See FIG. 10. A further cannula 82′ for venting the outer balloon can also be provided. The tubular portion 22′ is sized to contain the various cannulas. A pressure monitoring mechanism 50′, including a pressure gauge with visual indicator, pressure control valve, and stop valve, can be provided in a proximal portion of the tubular portion, which may be enlarged for ease of grasping by the user.

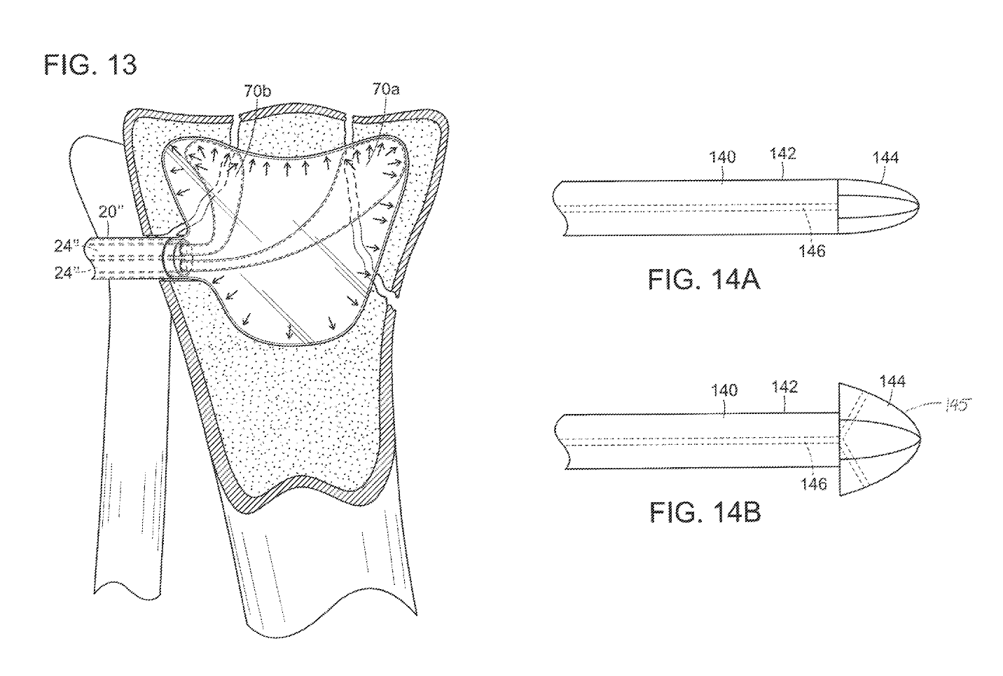

In a still further embodiment, in an outer balloon apparatus 20″, two or more cannulas 24″ for multiple inner balloons can be provided. Multiple inner balloon apparatuses can be provided. For example, FIG. 13 schematically illustrates a tibial plateau fracture in which two inner balloons 70a, 70b are employed to reduce the fracture. One balloon maintains reduction of the medial plateau, and the other balloon maintains reduction of the lateral plateau. The tibia is larger than the radius, so the use of two or more balloons can be useful for tibial fractures.

In some fractures, it is desirable to provide a stronger, more directed area of pressure to reduce a fracture. In this case, a probe apparatus can in inserted through the outer balloon apparatus in lieu or in addition to an inner balloon. The probe apparatus can be guided to provide pressure in a particular area. In one embodiment, referring to FIGS. 14A and 14B, the probe apparatus 140 includes a flexible sheath 142 and a distal tip element 144. The tip element is movable between a collapsed configuration (FIG. 14A), in which it can be inserted through a cannula in the outer balloon apparatus to the fracture site, and an expanded or deployed configuration (FIG. 14B). Within the fracture, the tip element 144 can be expanded to provide a contact surface 145, for example, a hemispherical or other more flattened surface, shaped to distribute pressure over a desired area. The tip element can include, for example, multiple overlapping plates or another umbrella-type structure that can be expanded upon actuation. For example, a cable actuating element 146 can be provided to expand the tip element. The flexible sheath element can be formed with a curve near the distal tip, for example, using a shape memory alloy. The user can rotate the sheath to direct the tip element toward the desired area. Cables can also be used to steer the apparatus 140 to a particular location. The outer surface or side wall of sheath 142 can also be inflated and collapsed to provide for easy removal. The tip element can be marked with a radiopaque marker, and the user can view the tip element in an image system, such as a fluoroscopic imaging system.

The device can be provided as a kit. FIG. 15 illustrates one embodiment of a kit 160, including an outer balloon apparatus 20, an inner balloon apparatus 60, and a selection of balloons, 30, 70, 70′, 70″, a probe 120, a probe apparatus 140, and stabilizing pins 110 and stabilizing bars 116.

The device and method are useful in treating fractures in osteoporotic bone, in which the fracture fragments often cannot be returned to their proper places. In more complex fractures, the present device can be tailored to directly reduce different aspects of the distal radius fracture pattern.

While this invention with reference to preferred embodiments thereof, it will be understood by those skilled in the art that various changes in form and details may be made therein without departing from the spirit and scope of the invention as has been particularly shown and described, except as indicated by the appended claims.

What is claimed is:

1. A system for assisting in the reduction of a fracture of a bone, comprising:
a delivery system comprising an introducer device, the introducer device configured to introduce an outer expandable member through an opening in the bone and introduce an inner expandable member through the opening within the outer expandable member to position a bone fragment relative to the bone.

2. The system of claim 1, wherein the introducer device comprises an outer introducer element configured to support the first expandable member, and an inner introducer element configured to support the inner expandable member.

3. The system of claim 2, wherein the outer introducer element further comprises an outer apparatus comprising a support element for mounting the outer expandable member, a passage through the support element for introducing a fluid into the outer expandable member when mounted on the support element.

4. The system of claim 2 wherein the inner introducer element further comprises an inner apparatus comprising a support element for mounting the inner expandable member, a passage through the support element for introducing a fluid into the inner expandable member when mounted on the support element, the inner introducer element configured to fit within at least a portion of the outer introducer element.

5. The system of claim 1, wherein the support element for the outer apparatus comprises a longitudinally extending tubular portion and a mounting element on a distal end of the tube configured to receive an outer balloon.

6. The system of claim 4, further comprising an inlet port for introducing a fluid into the passage.

7. The system of claim 6, further comprising a check valve adjacent the inlet port configured to prevent backflow through the inlet port.

8. The system of claim 4, further comprising a pressure monitoring mechanism in communication with the passage.

9. The system of claim 8, wherein the pressure monitoring mechanism includes a pressure gauge having a visual indicator on an exterior of the support element.

10. The system of claim 8, wherein the pressure monitoring mechanism further comprises a pressure relief valve configured to release fluid in the passage to ambient when a pressure value exceeds a determined pressure value.

11. The system of claim 3, wherein the outer apparatus further comprises a channel for supporting the inner balloon apparatus, a check valve disposed in the channel to prevent backflow through the channel.

12. The system of claim 4, wherein the inner apparatus comprises a longitudinally extending tube, and a mounting element on a distal end of the tube configured to receive the inner balloon.

13. The system of claim 12, further comprising an inlet port disposed for introducing a fluid into the tube.

14. The system of claim 3, further comprising a plurality of outer balloons and a plurality of inner balloons.

15. The system of claim 3, wherein the inner balloon is expanded to inflated state and is configured with a widened portion to provide pressure in a selected location.

16. The system of claim 1 further comprising a vacuum source coupled to the delivery system to remove fluid from an expanded member.

17. The system of claim 1 further comprising an anchor system to position the introducer relative to the distal radius of a patient.

18. The system of claim 17 wherein the anchor system further comprises one or more pins in bone elements of the patient.

19. The system of claim 1 further comprising a probe to be inserted in the outer expandable member to compress cancellous bone.

20. A kit comprising the system of claim 1 and further comprising an anchoring system.

21. A method for the reduction of a fracture of a bone, comprising:
introducing an outer balloon into the bone;
inflating the outer balloon to at least partially create a cavity within the bone;
introducing an inner expandable member into the bone;
inflating the inner member within the outer balloon to reduce the fracture;
introducing a bone-filling material into the cavity while maintaining reduction of the fracture with the inner expandable member.

22. The method of claim 21, wherein the outer balloon is deflated by rupturing the outer balloon.

23. The method of claim 21, wherein pressure within the outer balloon is monitored while the inner balloon is inflated.

24. The method of claim 21, wherein the inner member is manipulated within the bone to direct pressure on one or more displaced bone fragments to reduce an intra-articular fracture.

25. The method of claim 21, further comprising deflating the inner balloon and filling a cavity formed by the inner member deflation with a bone-filling material.

26. The method of claim 21, wherein the outer balloon is introduced in a fracture of a distal radius.

27. The method of claim 21, wherein the outer balloon is introduced along a fracture line of the fracture.

28. The method of claim 21, further comprising monitoring the reduction of the fracture by a radiographic imaging system.

29. The method of claim 28, wherein the radiographic imaging system comprises a fluoroscopic imaging system.

30. The method of claim 21, further comprising inserting an introducer through a single opening in a cortical bone.

31. The method of claim 21 further comprising anchoring an introducer device relative to a bone of a patient.

32. The method of claim 21 wherein the introducing step further comprises positioning an introducer device relative to a hole in a bone of a patient.

33. The method of claim 21 further comprising inserting a probe into the outer balloon to compress cancellous bone.

34. The method of claim 33 further comprising expanding a tip of the probe.

35. The method of claim 21 further comprising reducing a tibial fracture.

36. A method for the reduction of a fracture of a distal radius bone, comprising:
introducing an outer balloon into the bone;
inflating the outer balloon to at least partially create a cavity within the bone;
introducing a probe into the outer balloon to compress cancellous bone;
introducing a bone-filling material into the cavity while maintaining reduction of the fracture.

37. The method of claim 36, wherein an inner member is manipulated within the bone to direct pressure on one or more displaced bone fragments to reduce an intra-articular fracture.

38. The method of claim 37, further comprising inserting an inner balloon and subsequently deflating the inner balloon and filling a cavity remaining after deflation of the inner member with a bone-filling material.

39. The method of claim 36, wherein the outer balloon is introduced along a fracture line of the fracture.

40. The method of claim 36, further comprising inserting an introducer through a single opening in a cortical bone.

41. The method of claim 36 further comprising expanding a tip of the probe.

42. The method of claim 36 further comprising anchoring an introducer with an anchoring system.

43. The method of claim 42 wherein the anchoring step comprises a first pin and a second pin attached to bone elements.

44. The method of claim 36 further comprising steering the probe to position a tip of the probe within the distal radius.

45. The method of claim 36 further comprising inserting an outer balloon and an inner balloon through one or more introducer channels.
